(19) United States
(12) Patent Application Publication
(10) Pub. No.: US 2009/0124968 A1
(43) Pub. Date: May 14, 2009

(54) Self-Orientating Bifurcate Catheter

(75) Inventor: Justin Goshgarian, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/938,657

(22) Filed: Nov. 12, 2007

(51) Int. Cl. A61M 25/10

(52) U.S. Cl. 604/101.01; 604/284

(57) ABSTRACT

A bifurcate catheter having a self-orientating distal portion with improved pushability is disclosed. The distal portion of the bifurcate catheter includes a dilatation balloon attached to each of two distal branch portions of the catheter. The distal branch portions are of a length that permits rotational compliance of the distal portion of the bifurcate catheter as it is tracked over two guidewires and include at least one bond between the distal branch portions to improve pushability.

### FIELD OF THE INVENTION

The present invention relates in general to dilatation catheters employed in the treatment of vascular disease. More particularly, the present invention relates to a dilatation catheter assembly that has two balloons for treatment of vascular disease in arterial bifurcations.

### BACKGROUND OF THE INVENTION

Balloon angioplasty employs balloon tipped catheters to expand the walls of narrowed vessels and to deploy endoluminal prostheses to maintain lumen patency. Although systems and techniques exist that work well in many cases, no technique is applicable to every case. For example, special methods exist for dilating lesions that occur in branched or bifurcated vessels. A bifurcation is an area of the vasculature where a main vessel is bifurcated into two or more branch vessels. It is not uncommon for stenotic lesions to form in such bifurcations. The stenotic lesions can affect only one of the vessels, i.e., either of the branch vessels or the main vessel, two of the vessels, or all three vessels.

Methods to treat bifurcated vessels seek to prevent the collapse or obstruction of the main and/or branch vessel(s) during the dilation of the vessel to be treated. Such methods include techniques for using double guidewires and sequential percutaneous transluminal coronary angioplasty (PTCA) with stenting or the “kissing balloon” and “kissing stent” technique, which provide side branch protection.

Generally, PTCA is a procedure that involves passing a balloon catheter over a guidewire to a stenosis with the aid of a guide catheter. The guidewire extends from a remote incision to the site of the stenosis, and typically across the lesion. The balloon catheter is passed over the guidewire, and ultimately positioned across the lesion. Once the balloon catheter is appropriately positioned across the lesion, e.g., under fluoroscopic guidance, the balloon is inflated to break-up the plaque of the stenosis to thereby increase the vessel cross-section. The balloon is then deflated and withdrawn over the guidewire into the guide catheter to be removed from the body of the patient.

In many cases, a stent or other prosthesis must be implanted to provide permanent support for the vessel. When such a device is to be implanted, a balloon catheter, which carries a stent on its balloon, is deployed to the site of the stenosis. The balloon and accompanying stent are positioned at the location of the stenosis, and the balloon is inflated to circumferentially expand and thereby implant the stent. Thereafter, the balloon is deflated and the catheter and the guidewire are withdrawn from the patient.

Administering PTCA and/or implanting a stent at a bifurcation in a body lumen poses further considerations for the effective treatment of stenoses in the lumen. As mentioned above, dilating a vessel at a bifurcation may cause narrowing of an adjacent branch of the vessel. In response to such a challenge, attempts to simultaneously dilate both branches of the bifurcated vessel have been pursued. These attempts include deploying more than one balloon, more than one prosthesis/stent, a bifurcated or Y-shaped prosthesis/stent, or some combination of the foregoing. However, simultaneously deploying multiple and/or bifurcated balloons with or without endoluminal prostheses/stents, hereinafter individually and collectively referred to as a bifurcate catheter assembly, requires highly accurate placement of the assembly. Specifically, deploying a bifurcate catheter assembly requires positioning a main body of the catheter within the main vessel adjacent the bifurcation, and then positioning the independent distal portions of the catheter assembly into the branch vessels.

Tracking a bifurcated catheter assembly to a treatment site also presents additional considerations to the more standard PTCA procedure. For example, a bifurcated catheter must be tracked to the site as a unitary device until it reaches the bifurcation. However once the catheter reaches the bifurcated treatment site, each distal leg portion must be positioned within its respective branch of the vessel. Therefore, the catheter must be a unitary device during tracking and be a bifurcated device for treatment.

In order to achieve the foregoing objectives, two guidewires are typically required, one for placement of a portion of the bifurcate catheter assembly into each branch of the bifurcated vessel. Many devices known in the prior art have difficulty tracking and positioning a bifurcate catheter assembly utilizing two guidewires in an expeditious fashion due to entanglement of the guidewires, which often times may include a full twist of the guidewire distal portions together. Once such bifurcate catheters reach a “tangle” of the guidewires the clinician often times will attempt to push/force the catheter through, which can result in a hazardous twirling of the distal ends of the guidewires or in an impassable “knot” being created in the guidewires.

One approach to improve handling of a bifurcate catheter assembly as it is tracked over dual guidewires is to improve the rotational compliance of the distal end thereof by increasing the length of each section of catheter tubing distal of a “crotch” of the bifurcate catheter but proximal of each balloon. When such a bifurcate catheter reaches a twisting or other entanglement in the guidewires, the distal tubing sections may rotate, including fully about each other if necessary, to accommodate the entangled/crossed condition of the guidewires. However, while the increase in length of the distal tubing sections provides better rotational compliance, it impairs the pushability of the catheter due to the buckling of the distal tubing sections that can occur when the bifurcate catheter assembly meets resistance as it is being tracked through the vasculature. Therefore what is needed is a bifurcate catheter that allows for rotational compliance of its distal section as it is being tracked over dual guidewires without compromising its pushability.

### BRIEF SUMMARY OF THE INVENTION

Embodiments of the present invention are directed to bifurcate catheters that have a self-orientating distal portion with improved pushability. The bifurcate catheters include two dilation balloons extending from respective distal ends of distal branch portions of the catheter. The distal branch portions are of a length that permits rotational compliance as the bifurcate catheter is tracked over two guidewires and include at least one bond there between to improve pushability.

A bifurcate catheter according to an embodiment of the present invention includes a proximal shaft having a proximal end and a distal end with a first distal shaft branch and a second distal shaft branch that separately extend from the distal end thereof, such that the proximal ends of the first and second distal shaft branches meet or form a crotch or fork in the catheter shaft. A first balloon is attached to the first distal shaft branch and a second balloon is attached to the second distal shaft branch. At least one bond secures the first and second distal shaft branches together along the length thereof, wherein the at least one bond between the first and second distal shaft branches is located proximal of the proximal ends of the first and second balloons and distal of the distal end of the proximal shaft, viz., distal of the crotch or fork in the catheter shaft. The bond between the first and second distal shaft branches separates unattached longitudinal portions or lengths of the first and second distal shaft branches.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is an illustration of a self-orientating bifurcate catheter in accordance with an embodiment of the present invention.

FIG. 2 is an illustration of a self-orientating bifurcate catheter in accordance with another embodiment of the present invention.

FIG. 3 is an illustration of a self-orientating bifurcate catheter in accordance with another embodiment of the present invention.

FIG. 4 is an illustration of a distal end of a self-orientating bifurcate catheter according to another embodiment of the present invention.

FIG. 5 depicts the bifurcate catheter of FIG. 4 with a Y-shaped stent mounted thereon.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description.

Embodiments of the present invention are directed to bifurcate catheters that have a self-orientating distal portion with improved pushability. The bifurcate catheters include two dilation balloons extending from respective distal ends of distal branch portions of the catheter. The distal branch portions are of a length that permits rotational compliance as the bifurcate catheter is tracked over two guidewires and include at least one bond there between to improve pushability. “Rotational compliance” means that the each balloon is permitted to track over a respective guidewire and if a wrap in or twisting of the two guidewires is reached than the distal branch portions of the catheter are able to wrap or twist about each other, for example, proximal and/or distal of the bond there between, to accommodate the twisted guidewires and to permit the continued tracking of the balloons into a vessel bifurcation.

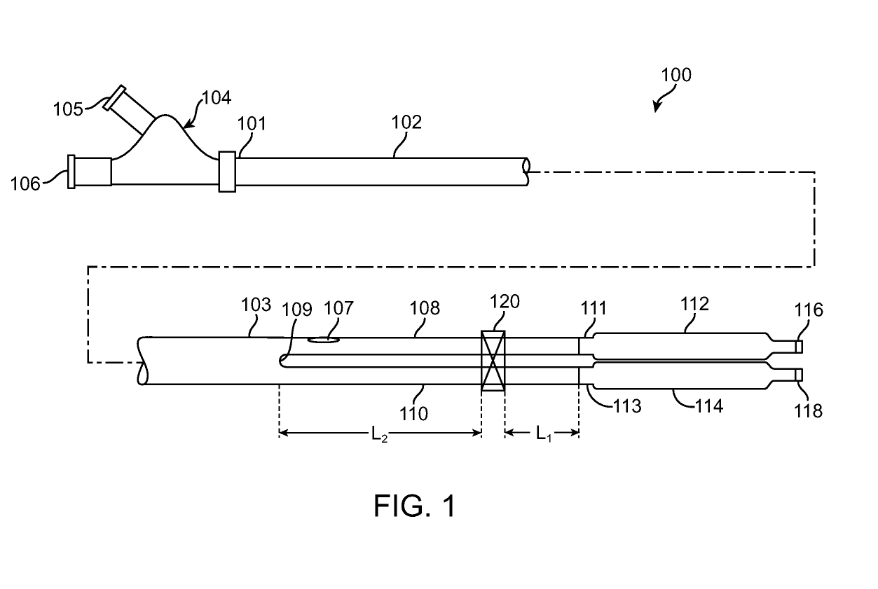

FIG. 1 is an illustration of self-orientating bifurcate catheter 100 in accordance with an embodiment of the present invention. Bifurcate catheter 100 has a first balloon 112 and a second balloon 114 and includes a proximal shaft 102 having a proximal end 101 attached to a hub 104 and a distal end 103 attached to first and second distal shaft branches 108, 110. Proximal ends of first and second distal shaft branches 108, 110 meet to form a crotch or fork 109 in the catheter shaft. One of first and second balloons 112, 114 is attached to a distal end of one of first and second distal shaft branches 108, 110, respectively.

Hub 104 includes a first guidewire port 106 that communicates with a first guidewire lumen (not shown) that extends through proximal shaft 102, second distal shaft branch 110 and second balloon 114 for receiving a first guidewire there through. A second guidewire port 107 is positioned along first distal shaft branch 108 and communicates with a second guidewire lumen (not shown) that extends through first distal shaft branch 108 and first balloon 112 for receiving a second guidewire there through. As such, bifurcate catheter 100 has functionalities associated with both over-the-wire and rapid-exchange catheters. Hub 104 also includes an inflation port 105 for coupling to a source of inflation fluid. Inflation port 105 fluidly communicates with each of first and second balloons 112, 114 via a main inflation lumen (not shown) that extends through proximal shaft 102 to fluidly communicate with a first branch inflation lumen (not shown) extending through first distal shaft branch 108 to first balloon 112 and a second branch inflation lumen (not shown) extending through second distal shaft branch 110 to second balloon 114.

In accordance with an embodiment of the present invention, each of first and second distal shaft branches 108, 110 is made of a length to achieve rotational compliance when bifurcate catheter 100 is tracked over first and second guidewires. Further, distal shaft branches 108, 110 are secured together by at least one tack or bond 120 that is a length “L₁” from proximal ends 111, 113 of balloons 112, 114, respectively and is a length “L₂” from crotch 109 of the catheter shaft. In an embodiment, length L₁ is between 0-15 cm and length L₂ is at least 5-20 cm. Tack or bond 120 allows the parallel, unattached portions of distal shaft branches 108, 110 that are defined by lengths L₁ and L₂ to freely rotate, as may be needed when bifurcate catheter 100 is tracked over two guidewires. With the addition of at least one bond 120 between first and second distal shaft branches 108, 110, the distal portion of bifurcate catheter 100 is rotationally compliant as well as more pushable for tracking through tortuous vasculature without buckling the distal sections.

In an embodiment, each of first and second distal shaft branches 108, 110 may be made of PEBAX, polyamides, polyethylenes, polyethylene terephthalate and polyurethanes and includes a tubular inner member (not shown) extending there through for providing the guidewire lumen. One inner member extends from proximate a distal tip 116 to second guidewire exit port 107 to provide the rapid-exchange functionality, while the other inner member extends from proximate a distal tip 118 to first guidewire port 106 to provide an over-the-wire functionality. In one embodiment, the inner member may be a tri-layer material having an outer layer of PEBAX, a tie layer of PLEXAR and an inner layer of high density polyethylene or HDPE. Proximal shaft 102 may be of PEBAX or other suitable material.

Bond or tack 120 may be made by placing a section of heat shrinkable tubing around the localized area(s) of the first and second distal shaft branches 108, 110 to be bonded and then applying a heat source, such as by laser, or hot box arrangement, to heat shrink the tubing there about. A bonding layer or filler of PEBAX, polyimide, polyamide, polyethylene, polyethylene terephthalate or polyurethane may be used between distal shaft branches 108, 110 in the bond area to assure adequate bonding between the branches without creating a kink or misalignment of their respective lumens. Alternative methods of bonding can be used such as radio-frequency, solvent, adhesive or mechanical bonding. The bond length should be as small as possible, for instance, a bond may be in the range of 0.5-1.0 mm in length.

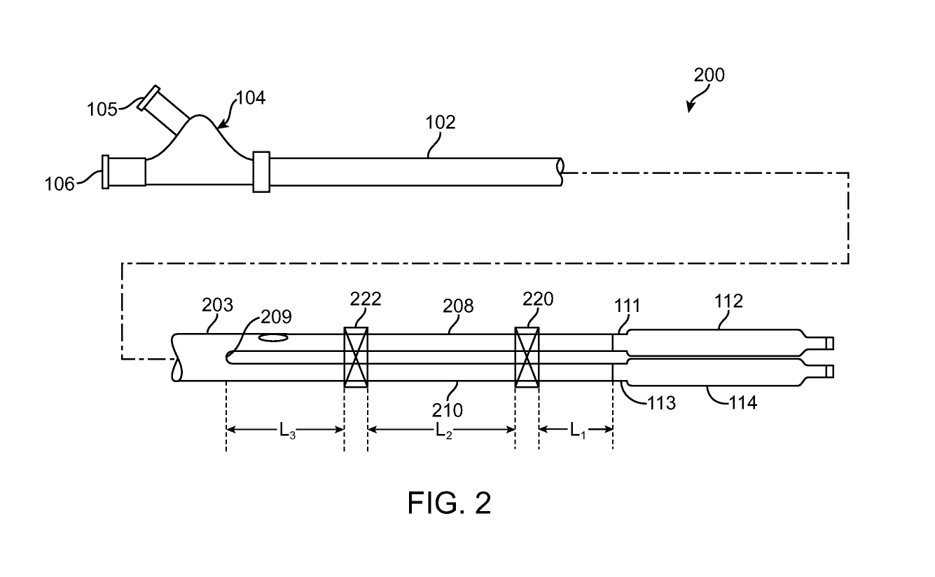

FIG. 2 is an illustration of a self-orientating bifurcate catheter 200 in accordance with another embodiment of the present invention with many of the same features as previously described with reference to the embodiment of FIG. 1. However in the embodiment of FIG. 2, bifurcate catheter 200 includes first and second distal shaft branches 208, 210 of a greater overall length than first and second distal shaft branches 108, 110 of bifurcate catheter 100. In addition, first and second distal shaft branches 208, 210 include a first and second bond 220, 222 there between. First bond 220 is a distance or length “L₁” from proximal ends 111, 113 of balloons 112, 114, respectively, whereas second bond 222 is a distance or length “L₂” from first bond 220. In turn, second bond 222 is a distance or length “L₃” from crotch or fork 209 of the catheter shaft. In an embodiment, unbonded or unattached lengths L₁, L₂ and L₃ of first and second distal shaft branches 208, 210 are between 0.5 and 15 cm in length. Tacks or bonds 220, 222 allow one or more of the unattached/unbonded portions of first and second distal shaft branches 208, 210 defined by lengths L₁, L₂ and L₃ to freely rotate, which allows the distal portion of bifurcate catheter 200 to be more rotationally compliant as it is tracked over two guidewires than the embodiment of FIG. 1.

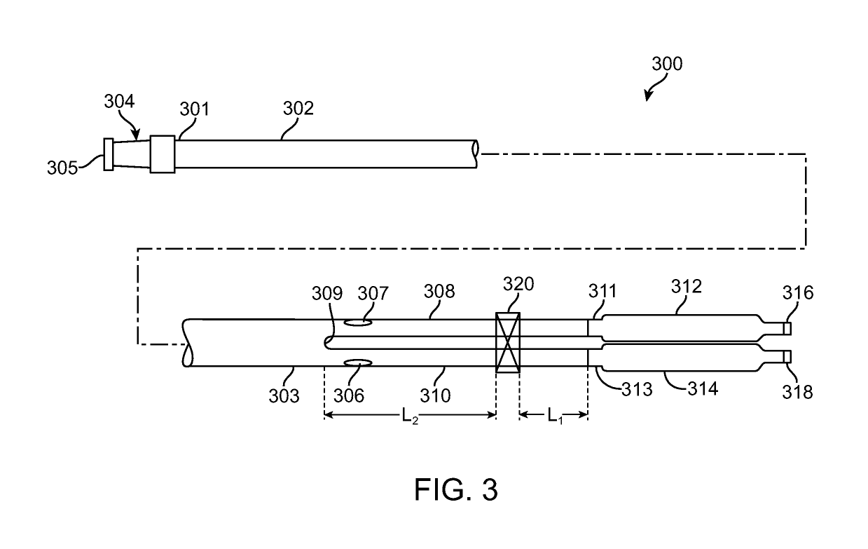

FIG. 3 is an illustration of self-orientating bifurcate catheter 300 in accordance with an embodiment of the present invention. Bifurcate catheter 300 has first balloon 312 and second balloon 314 with a single lumen proximal shaft 302 having a proximal end 301 attached to an inflation hub 304 and a distal end 303 attached to first and second distal shaft branches 308, 310. One of first and second balloons 312, 314 is attached to a distal end of one of first and second distal shaft branches 308, 310, respectively. Hub 304 includes an inflation port 305 for coupling bifurcate catheter 300 to a source of inflation fluid. Inflation port 305 fluidly communicates with each of first and second balloons 312, 314 via a main inflation lumen (not shown) that extends through proximal shaft 302 to fluidly communicate with a first branch inflation lumen (not shown) extending through first distal shaft branch 308 to first balloon 312 and a second branch inflation lumen (not shown) extending through second distal shaft branch 310 to second balloon 314.

In the embodiment of FIG. 3, a first guidewire port 306 is positioned along distal shaft branch 310 and communicates with a first guidewire lumen (not shown) that extends through second distal shaft branch 310 and second balloon 314 for receiving a first guidewire there through. A second guidewire port 307 is positioned along first distal shaft branch 308 and communicates with a second guidewire lumen (not shown) that extends through first distal shaft branch 308 and first balloon 312 for receiving a second guidewire there through. As such, bifurcate catheter 300 has dual rapid-exchange functionalities.

In accordance with an embodiment of the present invention, each of first and second distal shaft branches 308, 310 is made of a sufficient length to achieve rotational compliance when bifurcate catheter 300 is tracked over first and second guidewires. Further, first and second distal shaft branches 308, 310 are secured together by at least one bond 320 that is of a length “L₁” from proximal ends 311, 313 of balloons 312, 314, respectively and is of a length “L₂” from fork or crotch 309 of the catheter shaft. In an embodiment, length L₁ is between 0-15 cm and length L₂ is at least 0-15 cm. As in the embodiment of FIG. 1, bond 320 allows the unbonded or unattached portions of distal shaft branches 308, 310 that are defined by lengths L₁ and L₂ to freely rotate about each other, as may be needed when bifurcate catheter 300 is tracked over two guidewires. With the addition of at least one bond 320 between first and second distal shaft branches 308, 310, the distal portion of bifurcate catheter 300 is rotationally compliant as well as pushable for tracking through tortuous vasculature without buckling.

In an embodiment, each of first and second distal shaft branches 308, 310 may be made of PEBAX and includes a tubular inner member (not shown) extending there through for providing the guidewire lumen. One inner member extends from proximate distal tip 316 to second guidewire exit port 307 to provide a first rapid-exchange functionality, while the other inner member extends from proximate distal tip 318 to first guidewire port 306 to provide a second rapid-exchange functionality. In an embodiment, the inner member may be a tri-layer material having an outer layer of PEBAX, a tie layer of PLEXAR and an inner layer of HDPE. Proximal shaft 302 may be a single-lumen metallic hypotube or of a polymeric tubing of PEBAX, for example, with a transition area proximate distal end 303 for splitting the inflation lumen into communication with each of the inflation lumens of distal shaft branches 308, 310.

In addition to the hybrid over-the-wire and rapid-exchange bifurcate catheters 100, 200 and the dual rapid-exchange bifurcate catheter 300 disclosed above, a dual over-the-wire bifurcate catheter, a dual fixed wire bifurcate catheter as well as a hybrid over-the-wire and fixed wire bifurcate catheter may be modified in accordance with the aforementioned embodiments of the present invention as would be apparent to one of ordinary skill in the art. Thus the disclosed bifurcate catheter embodiments are not meant to limit the application of the present invention.

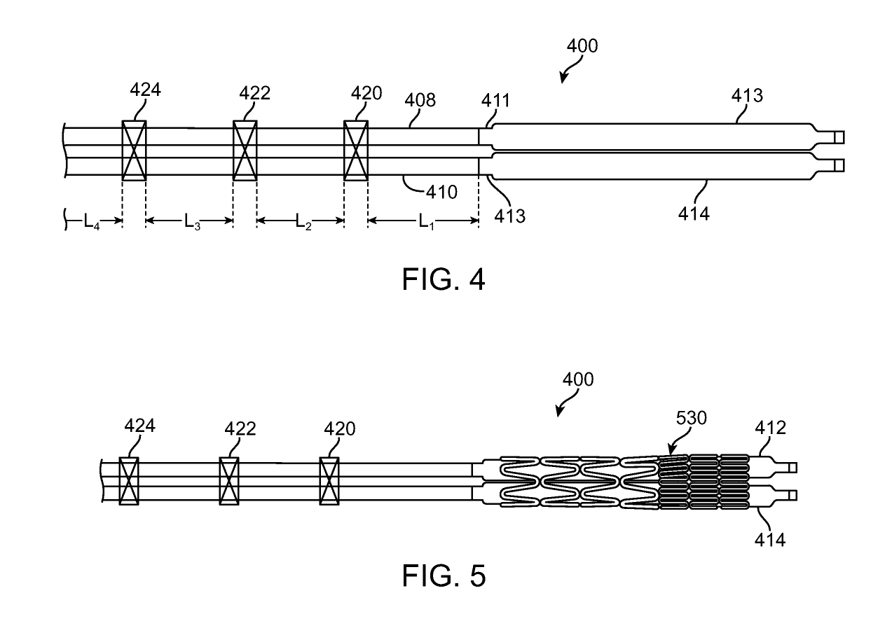

FIG. 4 is an illustration of a distal end of a self-orientating bifurcate catheter 400 according to another embodiment of the present invention and may be any type of bifurcate catheter mentioned in the preceding paragraphs. Bifurcate catheter 400 includes first, second and third bonds 420, 422, 424 for securing distal shaft branches 408, 410 together. First bond 420 is a distance or length “L₁” from proximal ends 411, 413 of balloons 412, 414, respectively. Second bond 422 is a distance or length “L₂” from first bond 420 and a distance or length “L₃” from third bond 424. In turn, third bond 424 is a distance or length “L₄” from a crotch (not shown) of catheter 400. In an embodiment, each of the unbonded or unattached portions of distal shaft branches 408, 410 defined by lengths L₁, L₂, L₃ and L₄ is between 0.5 and 15 cm. In another embodiment, at least one of the unbonded portions defined by lengths L₁, L₂, L₃ and L₄ is at least 15 cm. Although in FIG. 4 each of the unbonded lengths L₁, L₂, and L₃ appears to be the same length, this need not be the case and each may be of the same or different length from the others depending on the rotational compliance to be achieved.

As in the previous embodiments, tacks or bonds 420, 422, 424 allow one or more of the unattached/unbonded portions of distal shaft branches 408, 410 defined by lengths L₁, L₂, L₃ and L₄ to freely rotate about each other, which allows the distal portion of bifurcate catheter 400 to be rotationally compliant as it is tracked over two guidewires.

FIG. 5 depicts bifurcate catheter 400 of FIG. 4 with a Y-shaped or bifurcate stent 530 mounted thereon, which may be any bifurcate stent known in the art such as the stents shown or described in U.S. Pat. No. 6,129,738 to Lashinski et al., which is incorporated by reference herein in its entirety.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A bifurcate catheter comprising:
a proximal shaft having a proximal end and a distal end;
a first distal shaft branch and a second distal shaft branch, wherein the first and second distal shaft branches separately extend from the distal end of the proximal shaft forming a crotch in the catheter shaft;
a first balloon attached to the first distal shaft branch;
a second balloon attached to the second distal shaft branch; and
at least one bond that secures the first and second distal shaft branches together, wherein the at least one bond between the first and second distal shaft branches is located proximal of the proximal ends of the first and second balloons and distal of the crotch in the catheter shaft.

2. The bifurcate catheter of claim 1, wherein the bond between the first and second distal shaft branches separates unattached portions of the first and second distal shaft branches.

3. The bifurcate catheter of claim 2, wherein a first length L₁ of unattached portions of the first and second distal shaft branches extends between the bond and the proximal ends of the first and second balloons.

4. The bifurcate catheter of claim 3, wherein the first length L₁ of unattached portions of the first and second distal shaft branches are rotatable about one and other.

5. The bifurcate catheter of claim 3, wherein the first length L₁ is between 0.5 and 20 cm.

6. The bifurcate catheter of claim 3, wherein a second length L₂ of unattached portions of the first and second distal shaft branches extends between the bond and the crotch in the catheter shaft.

7. The bifurcate catheter of claim 6, wherein the second length L₂ of unattached portions of the first and second distal shaft branches are rotatable about one and other.

8. The bifurcate catheter of claim 1, wherein the at least one bond that secures the first and second distal shaft branches together comprises a first bond and a second bond such that the first bond and the second bond are separated by unattached portions of the first and second distal shaft branches.

9. The bifurcate catheter of claim 8, wherein a first length L₁ of unattached portions of the first and second distal shaft branches extends between the first bond and the proximal ends of the first and second balloons.

10. The bifurcate catheter of claim 9, wherein the first length L₁ of unattached portions of the first and second distal shaft branches are rotatable about one and other.

11. The bifurcate catheter of claim 9, wherein the first length L₁ is between 0.5 and 20 cm.

12. The bifurcate catheter of claim 9, wherein a second length L₂ of unattached portions of the first and second distal shaft branches extends between the first bond and the second bond.

13. The bifurcate catheter of claim 12, wherein the second length L₂ of unattached portions of the first and second distal shaft branches are rotatable about one and other.

14. The bifurcate catheter of claim 9, wherein a third length L₃ of unattached portions of the first and second distal shaft branches extends between the second bond and the crotch in the catheter shaft.

15. The bifurcate catheter of claim 13, wherein the third length L₃ of unattached portions of the first and second distal shaft branches are rotatable about one and other.
